(19) United States
(12) Patent Application Publication
(10) Pub. No.: US 2024/0194468 A1
(43) Pub. Date: Jun. 13, 2024

(54) HYDROGEN FLUORIDE DETECTION USING MASS SPECTROMETRY

(71) Applicant: Syft Technologies Ltd, Christchurch (NZ)

(72) Inventors: Caleb Allpress, Christchurch (NZ); Richard Butler, Christchurch (NZ)

(21) Appl. No.: 18/581,486

(22) Filed: Feb. 20, 2024

Related U.S. Application Data
(63) Continuation of application No. PCT/IB2022/057747, filed on Aug. 18, 2022.
(60) Provisional application No. 63/235,750, filed on Aug. 22, 2021.

(51) Int. Cl. H01J 49/04; G01N 33/00

(52) U.S. Cl. CPC H01J 49/0422; G01N 33/0027

(57) ABSTRACT

Systems and methods are described that facilitate effective detection and quantification of hydrogen fluoride within a sample utilizing mass spectrometry (MS). A MS analysis is performed on a gas sample to obtain an unfiltered set of MS signal data. Hydrogen fluoride (HF) is removed from the gas sample by filtration to form a HF filtered gas sample, and MS analysis is performed on the HF filtered gas sample to obtain a filtered set of MS signal data. The filtered set of MS signal data is compared with the unfiltered set of MS signal data, and a presence and/or concentration of HF within the gas sample is determined based upon the comparison.

### CROSS-REFERENCE TO RELATED APPLICATIONS

This application is a continuation of PCT International Application No. PCT/IB2022/057747, filed Aug. 18, 2022, entitled “Hydrogen Fluoride Detection Using Mass Spectrometry,” which claims priority to U.S. Provisional Patent Application No. 63/235,750, filed Aug. 22, 2021, and entitled “Hydrogen Fluoride Detection Using Mass Spectrometry,” the disclosures of which are incorporated herein by reference in their entireties.

### FIELD

The present invention relates to detection of gaseous components using mass spectrometric analysis methods.

### BACKGROUND

Mass spectrometry has been a long-standing analytical technique for gas analysis with applications across a diverse range of industries. The wide-spread application of mass spectrometry is largely due to its ability to quantify a wide range of analytes that include volatile organic and inorganic compounds with a high degree of specificity. The specificity of mass spectrometry can be achieved by control of the analyte ionization mechanism, and by precise measurement of an analyte ion's mass-to-charge ratio.

Selected Ion Flow Tube-Mass Spectrometry (SIFT-MS) is a form of direct chemical ionization mass spectrometry that has the advantages of a very soft ionization mechanism as well as the use of up to 8 different reagent ions to provide multiple ionization channels for a single analyte. This combination of features allows for real-time analysis with low detection limits and generally high selectivity when compared to other real time MS techniques.

Across the trace gas analysis industry, it is challenging to measure hydrogen fluoride (HF) with high selectivity and low detection limits while retaining simultaneous measurement capabilities for other compounds. For example, SIFT-MS can quantify HF by utilizing soft-ionization reactions with several different reagent ions, including (without limitation)O⁻ and OH⁻, which both react with HF by a proton transfer mechanism to form the product ion F⁻ at m/z=−19. Other compounds that react with these reagent ions to form a fluoride ion will cause a spectral interference with HF and include many classes of fluorine-containing compounds including perfluorocarbons (PFCs), hydrofluorocarbons (HFCs), perfluoroalkyl amines, and inorganic fluorides. The efficiency of fluoride ion generation for many of these potential interferents is typically low. However, due to the properties of these compounds (many of which are used in heat transfer systems), such compounds may be present in bulk quantities in fabrication environments (e.g., within clean rooms of semiconductor fabrication facilities). When trace levels of HF need to be reliably quantified, the contributions of these interferent compounds can be significant. Therefore, the selective quantification of hydrogen fluoride relies on an approach that accounts for the contribution of these other compounds to the m/z=−19 signal.

In SIFT-MS analysis, spectral interferences from known compounds with unique product ions are typically removed by subtraction. This approach works well when interferent compounds are limited in number and can be well-characterized. However, in a matrix where there is the potential presence of several different fluorinated compounds, this process cannot be pragmatically applied as it requires significant measurement time which comes at the cost of overall detection limits. Additionally, in matrices where not all interferences are known or no other product ions are viable for subtraction, subtraction will not remove all possible false positives. For example, in a semiconductor fabrication environment, there are both unknown fluorine-containing compounds present and compounds such as nitrogen trifluoride (NF₃) that do not produce unique product ions compared to HF, the subtraction approach is not viable. As a result, deployment of SIFT-MS in semiconductor fabrication environments have not provided a satisfactory solution to date due to an unacceptably high level of false positive HF events.

With the previously noted issues in mind, it is challenging for the gas analysis industry to measure hydrogen fluoride (HF) with high selectivity and low detection limits while retaining simultaneous measurement capabilities for other compounds. It is often important to provide an accurate presence determination and measurement of HF concentration in certain industrial and/or manufacturing environments (e.g., in clean rooms of semiconductor manufacturing/fabrication facilities). Hydrogen fluoride has a simple molecular structure with a single bond between hydrogen and fluoride. The quantification of HF is typically based on a non-specific fluoride ion (F⁻) measurement. In other words, the F⁻ measurement contains no information about the bond from where it was previously present (could be HF or some other fluoride compound). This results in a lack of confidence that the qualification and quantification measurement is in fact HF and not another fluorinated compound.

It is therefore desirable to provide a mass spectrometric configuration that accurately identifies the presence and amount of HF in a particular environment.

### SUMMARY

In accordance with example embodiments of the invention, a method for detection of hydrogen fluoride (HF) within a gas sample comprises performing mass spectrometry (MS) analysis on a gas sample to obtain an unfiltered set of MS signal data, removing hydrogen fluoride (HF) from the gas sample by filtration to form a HF filtered gas sample, performing mass spectrometry (MS) analysis on the HF filtered gas sample to obtain a filtered set of MS signal data, comparing the filtered set of MS signal data with the unfiltered set of MS signal data, and determining a presence and/or concentration of HF within the gas sample based upon the comparison.

In other example embodiments, a mass spectrometry (MS) detection system with hydrogen fluoride (HF) specificity comprises a MS detector to analyze a gas sample and determine presence and/or concentration of a plurality of chemical species within the gas sample, at least one sample flow line to facilitate flow of the gas sample from a sample source at a selected environment to an inlet of the MS detector, and a selective hydrogen fluoride scrubber coupled with the sample flow line, where the scrubber filters hydrogen fluoride (HF) from the gas sample prior to delivery to the inlet of the MS detector. The system is configured to selectively permit a switch between a first flow of unfiltered gas sample to the inlet of the MS detector and a second flow of HF filtered gas sample to the inlet of the MS detector.

In further example embodiments, a mass spectrometry (MS) detection system with hydrogen fluoride (HF) specificity comprises a MS detector to analyze a gas sample to determine presence and/or concentration of chemical species within the gas sample, a sample flow line that provides the gas sample from a selected environment to the MS detector, the sample flow line including a diverter to selectively divert the gas sample between a first sample line and a second sample line, where each of the first and second sample lines provide the gas sample to a MS inlet of the MS detector, and a selective hydrogen fluoride scrubber (SHFS) provided in the second sample line, where the SHFS filters hydrogen fluoride (HF) from the gas sample prior to delivery to the inlet of the MS detector. The system further comprises a controller that selectively controls the MS detector so as to facilitate delivery and analysis of the gas sample to the MS inlet in an unfiltered state via the first sample line and delivery and analysis of the gas sample to the MS inlet in a filtered state via the second sample line, wherein the controller further facilitates a determination of HF presence and/or HF concentration within the gas sample based upon a comparison of MS analysis of the sample gas in the unfiltered state and MS analysis of the sample gas in the filtered state.

The above and still further features and advantages of the present invention will become apparent upon consideration of the following detailed description of specific embodiments thereof.

### BRIEF DESCRIPTION OF THE DRAWINGS

FIG. 1 depicts an example embodiment of HF specific mass spectrometric system in accordance with the present invention.

FIG. 2 depicts a flowchart of operational features for an example embodiment in accordance with the present invention.

FIG. 3 depicts mass spectrometry signal plots of a sample obtained by the system of FIG. 1 with and without HF filtration from the sample.

FIG. 4 is a plot of signal data obtained by the system of FIG. 1 of a sample including HF and other fluorine interfering species, in which concentration of HF is determined without HF filtration.

FIG. 5 is a plot of signal data obtained by the system of FIG. 1 of the same sample for FIG. 4, in which concentration of HF is determined with HF filtration and according to methods described herein.

Like reference numerals have been used to identify like elements throughout this disclosure.

### DETAILED DESCRIPTION

In the following detailed description, while aspects of the disclosure are disclosed, alternate embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising,” “including,” “having,” and the like, as used with respect to embodiments of the present disclosure, are synonymous.

In accordance with example embodiments, systems and methods are described herein that facilitate accurate and reliable detection and quantification of hydrogen fluoride (HF) in a sample, particularly when the sample includes other interfering fluoride species and/or other (e.g., non-fluorine) compounds that can interfere with HF detection and quantification. The systems and methods provide for mass spectrometric analysis of a sample and also a modified sample in which HF is selectively removed (e.g., via a scrubber/filtration) from the sample. The sample and modified (HF removed) samples are compared to determine whether and to what extent HF is present within the sample.

The use of a chemical scrubber or filtration unit that selectively removes the highly reactive HF molecule from a sample stream, but not the less reactive compounds that cause spectral interferences, provides an additional and more selective method for HF quantification by MS techniques such as SIFT-MS. This approach does not require detailed knowledge of all the interfering compounds present in the sample stream to selectively report an HF concentration. A viable scrubber substrate is described herein that removes HF from a sample gaseous stream but allows the majority of other fluorine-containing compounds to pass through the scrubber substrate. The scrubber substrate allows HF to be selectively quantified by monitoring the difference between a scrubbed/filtered and non-scrubbed/unfiltered sample stream.

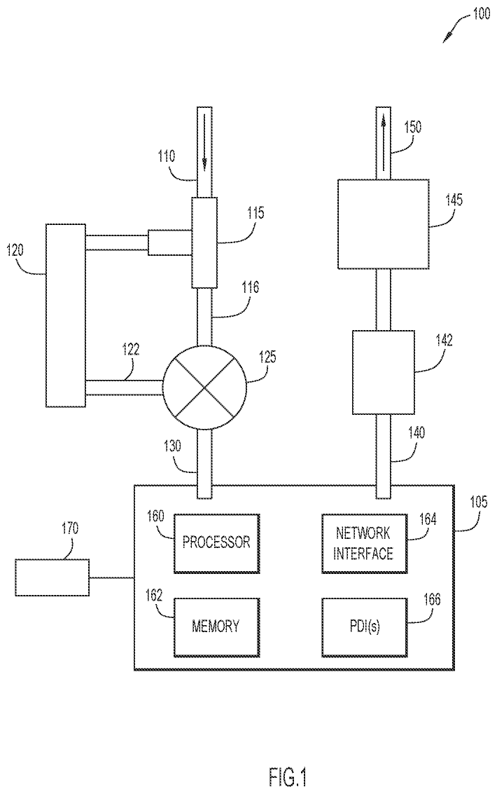

An example and nonlimiting embodiment of a mass spectrometric detection system is depicted in FIG. 1. The detection system 100 comprises a mass spectrometry detector 105 that receives a gas sample and performs mass spectrometry analysis of the sample to determine chemical compounds/chemical species within the sample by ionization of such compounds performing identification/quantification analysis based upon the mass-to-charge ratio of the ionized compounds.

An example embodiment of a detector 105 that can be implemented for use in the system 100 is a mass spectrometry system that utilizes Selected Ion Flow Tube-Mass Spectrometry (SIFT-MS). A non-limiting example SIFT-MS mass spectrometry detector that can be used in the system is a Voice200infinity mass spectrometer detector commercially available from Syft Technologies (New Zealand). The SIFT-MS detector allows mass spectrometry to be applied in applications requiring real-time analysis with low detection limits, e.g., to the parts-per-billion (ppb) and parts-per-trillion (ppt) levels.

A gas sample flow line 110 receives an input gas from a sample source and directs the gas to an inlet 130 of the detector 105 in a manner as described herein. A carrier gas (e.g., ultra-high purity helium or nitrogen) can be utilized to transport the sample within the system. The sample source can be a processing room (e.g., clean room) of a semiconductor manufacturing or fabrication facility that manufactures or processes one or more semiconductor components. In such facilities, hydrogen fluoride (HF) can be used (e.g., as an etchant) and can be present within the room. The gas sample flow line 110 can selectively acquire a gas sample from the room and deliver the sample to the system 100.

Disposed within the gas sample flow line 110 is a diverter comprising a tee member 115 and a 3-way valve 125. The diverter is provided to selectively divert flow of the gas sample from the sample source to the detector inlet 130 so as to either bypass a filter or scrubber or facilitate passage through the scrubber as described herein. The tee member 115 (e.g., a PFA plastic tee fitting) splits the sample from flow line 110 into two flowing sample streams that flow through sample lines 116, 122. A first stream flowing within a first sample flow line 116 proceeds from the tee member 115 directly to the detector inlet 130, while a second stream from a second sample flow line 122 is first directed from the tee member 115 through a scrubber or filter 120 prior to being directed to the detector inlet so as to provide a filtered sample to the detector for analysis. A 3-way valve 125 is provided in-line with and at the outlets for each of the first sample flow line 116 and the second sample flow line 122. The valve 125 can be controlled (e.g., automatically controlled via a controller/processor of the detector 105 as described herein) so as to selectively provide either a first, unfiltered sample stream from the first sample flow line 116 or the second, filtered sample stream from the second sample flow line 122 to the detector inlet 130.

Any other suitable fitting or combination of fittings or other structure can also be provided to selectively divert the gas sample into two or more (e.g., multiple) sample streams to accommodate filtering and/or other types of processing of the sample prior to analysis by the detector. For example, while the 3-way valve 125 is depicted in the system 100 of FIG. 1 as being located downstream from the diverter/tee member 115, other embodiments for the system are also possible in which the positioning of the 3-way valve 125 and tee 115 are reversed (i.e., the 3-way valve is upstream from the tee). Further, the system 100 depicts a single gas sample flow line that directs a flow of the gas sample from the gas sample source to the system. Alternatively, two separate and independent sample flow lines, with one sample flow line not including any scrubber or filter and the other sample flow line including the scrubber or filter, can be provided from the sample source (e.g., a manufacturing or processing room for semiconductor fabrication) to the detector, where each sample flow line can be adjusted via one or more valves to selectively permit or prevent flow into the detector (thus facilitating flow of filtered or unfiltered gas sample to the detector).

A reagent ion generator/reagent ion supply source 170 generates and delivers any selected number and types of reagent ions (e.g., NO⁺, O₂⁺, H₃O⁺, O⁻, O₂⁻, OH⁻, NO₂⁻ and/or NO₃⁻) in a carrier medium (e.g., nitrogen) within a reaction chamber within the detector, where the reaction chamber also receives the sample delivered from the detector inlet 130. For example, the reagent ion generator and supply source for a SIFT-MS detector (e.g., a Voice200infinity mass spectrometer detector as described herein) can comprise a microwave discharge component that generates reagent ions of the types noted herein from a gas supply mixture of air or oxygen (O₂), water (H₂O) and nitrogen (N₂) (where the gas supply also functions as a carrier medium for the reagent ions formed). The SIFT-MS detector further includes a quadrupole filter to selectively deliver one or more types of the generated reagent ions for entry into the reaction chamber (while preventing others from entering the reaction chamber). This facilitates exposure and selective reaction (utilizing SIFT-MS) within the reaction chamber of the detector of the one or more selected reagent ions with one or more chemical compounds within the sample to form reaction products. For example, OH⁻ ions selectively permitted to enter the reaction chamber within the MS detector react with HF within the gas sample to generate one or more ionization products that are detectable, identifiable and quantifiable by MS analysis within the detector.

The detector 105 performs mass spectrometry analysis of the sample including reaction products (from the reaction of one or more reagent ions with chemical compounds in the sample) and then outputs the analyzed sample via an outlet 140 and through a flow line 150. A pump 145 can be provided in-line along the flow line 150 to direct the analyzed sample (for collection or to a selected exhaust system). A back pressure regulator 142 can also be provided in the flow line 150 to control pressure and flow of the sample into and through the detector.

The detector 105 can include a controller or processor 160 and memory 162 to store any one or more applications for processing data collected by the detector during the sample analysis. The processor 160 can comprise a microprocessor that executes control process logic instructions stored within the memory 162, including operational instructions and one or more software applications stored within such memory, where the processor 160 (utilizing the one or more software applications) performs operations in accordance with the operational/method steps described herein for mass spectrometry and analysis of gas samples.

The memory 162 of the detector 105 can include a library of data including known (i.e., “fingerprint”) m/z signal data for analyses of various specific chemical components based upon a particular application. Alternatively, or in addition to a data library provided in the memory 162 of the detector 105, the detector 105 can further include a suitable network interface 164 that facilitates communications and exchange of data between the detector 105 and other computing systems over any suitable type of wired and/or wireless network (e.g., any one or more of local or wide area networks, Internet Protocol (IP) networks such as intranet or internet networks, telephone networks (e.g., public switched telephone networks), wireless or mobile phone or cellular networks, and any suitable combinations thereof). The detector 105 can further include any one or more suitable types of peripheral device interfaces (PDIs) 166 that facilitate a hardwire connection or other coupling (e.g., wireless) with the detector (e.g., keyboard, display, mouse device, microphone device, audio device, etc.) so as to facilitate exchange of data (I/O operations) between the detector and an operator of the detector or other computer device.

The memory 162 of the detector can comprise one or more computer readable storage media that may further comprise read only memory (ROM), random access memory (RAM), magnetic disk storage media devices, optical storage media devices, flash memory devices, electrical, optical and/or other physical/tangible (e.g., non-transitory) memory storage devices, and any combinations thereof. In other words, the one or more computer readable storage media are one or more physical, tangible hardware devices that that can retain and store instructions for use by the detector, for example including software comprising computer executable instructions operable to perform certain operations when the software is executed. A computer readable storage medium (or one or more computer readable storage media), as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The scrubber or filter 120 for the system 100 is configured to selectively remove HF from a sample passing through the filter while allowing other chemical components, including most or substantially all other fluoride species, to remain in the sample for analysis by the detector 105. Any suitable filter material that is capable of selectively scrubbing or removing HF from a gas sample with little or no removal of other fluoride and/or other components may be suitable for certain applications.

In example embodiments, the filter material comprises a silica-based scrubber substrate in the form of fibrous material or a material comprising an entangled mixture or bundle of silica fibers (e.g., silica wool) with a suitably large number of active sites that react selectively and irreversibly with acidic functional groups. In a non-limiting example, the scrubber substrate material comprises glass wool fibers having dimensions of no greater than about 4 cm in length and transverse cross sections (e.g., diameters) ranging from about 15 micrometers (microns) to about 25 microns. The bulk density of the material can range from about 20 kg/m³ to about 160 kg/m³. The material can be pretreated by heating to a suitable temperature before use (e.g., about 80° C. for a period of about 6 hours). This scrubber substrate material has been found to meet certain criteria for effectively filtering of HF: effectively removes HF removal at high flow rates, high transmission rates allowed for other fluoride compounds, scrubber substrate is effective for a long period of time (long lifetime), it is easy to handle and manufacture, it has a low pressure drop and a large surface area to volume ratio (high sample gas to surface interaction to reduce filter volume to increase response time). However, as previously noted, any other suitable material (e.g., silica beads and/or any other form of filtration material) capable of effectively trapping and removing HF from a sample can be used in the systems and methods described herein.

The scrubber substrate can effectively remove up to 50 ppbv HF with an efficiency of greater than 99%. In addition, twelve fluorine-containing compounds, which cover four main compound classes (polyfluorinated hydrocarbons, perfluorocarbons, perfluoroalkylamines, and inorganic fluorides) and cover a range of chemical properties (boiling points, molecular weights, and polarities), can be present in a gas stream and further substantially remain in the gas stream after having been processed by the scrubber substrate. These types of fluorine-containing compounds can be present along with HF, e.g., in gases sampled from clean rooms and/or other processing locations within a semiconductor manufacturing or fabrication facility.

In an example embodiment, a scrubber substrate comprising silica wool was tested, with results shown in Table 1 below. Eleven of these fluorine-containing compounds were found to have transmission rates of greater than 99% when passed through the scrubber substrate (e.g., less than 1% by weight of fluorine-containing compound is trapped by and prevented from passing through the scrubber substrate), indicating a very low interference when determining presence and quantitation of HF according to the methods described herein. The only fluorine-containing compound that provided some level of interference was HFBA (heptafluorbutyric acid). However, this compound can still be compensated for and addressed based upon awareness of the potential issue it presents when utilizing the methods described herein. For example, HFBA, when present, can be accounted for by subtraction with a selective product ion so as to substantially prevent interference HF detection and quantitation.

TABLE 1
Twelve Fluorinated Interference Compounds Tested for
Transmission Through Selective HF Scrubber Substrate

| | Molecular | Boiling | Per Compound | |
|---|---|---|---|---|
| | Weight | Point | Transmission (%) | |
| Compound | (g mol⁻¹) | (° C.) | Mean | SD |
| Nitrogen trifluoride | 71.0 | −129.0 | 99.5 | 1.1 |
| Carbon tetrafluoride | 88.0 | −127.8 | N/A | N/A |
| Trifluoromethane | 70.0 | −82.1 | 99.6 | 1.1 |
| Difluoromethane | 52.0 | −52 | 99.9 | 3.6 |
| Octafluorocyclobutane | 200.0 | −5.8 | 99.6 | 0.9 |
| Perfluorotributylamine | 671.1 | 178 | 99.5 | 6.1 |
| Heptafluorobutyric | 214.0 | 120 | 31.7 | 1.9 |
| acid | | | | |
| Hexafluoro-1,3- | 162.0 | 5.5 | 99.6 | 3.2 |
| butadiene | | | | |
| 2,3,3,3- | 114.0 | −30 | 99.6 | 1.2 |
| Tetrafluoropropene | | | | |
| 1,2,4,5- | 150.1 | 90 | 99.7 | 0.8 |
| Tetrafluorobenzene | | | | |
| Hexafluorobenzene | 186.1 | 80.1 | 99.5 | 1.2 |
| Octafluorotoluene | 236.1 | 104 | 99.4 | 1.4 |

The scrubber substrate can also be configured (e.g., by selection of suitable packing and particle or fiber sizes of the materials forming the substrate) such that the flow rate of sample is not significantly impacted when the sample is directed for flow through the filter. In an example in which a flow rate of unfiltered sample through the detector is set (e.g., by suitable control of pump and back pressure regulator parameters) at about 2 L/minute, the scrubber substrate can be configured such that the flow rate of the filtered sample is no more than 600 mL/minute less than the flow rate of the unfiltered sample. The effect of the flow rate can be diminished to a greater extent when utilizing shorter substrate (e.g., glass) fibers, such as fibers no greater than about 4 cm (e.g., 3 cm or less).

Thus, the scrubber substrate, when implemented by the system 100 by diverting sample flow via tee 110, allows for selective removal of HF (and primarily or substantially only HF) from the sample without significantly impacting sample flow to the detector during system operations.

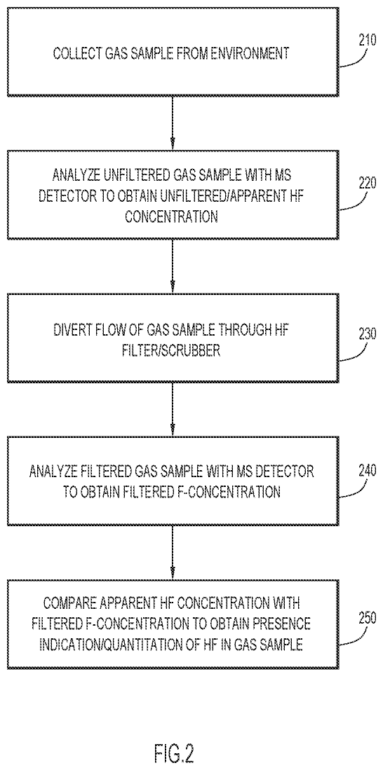

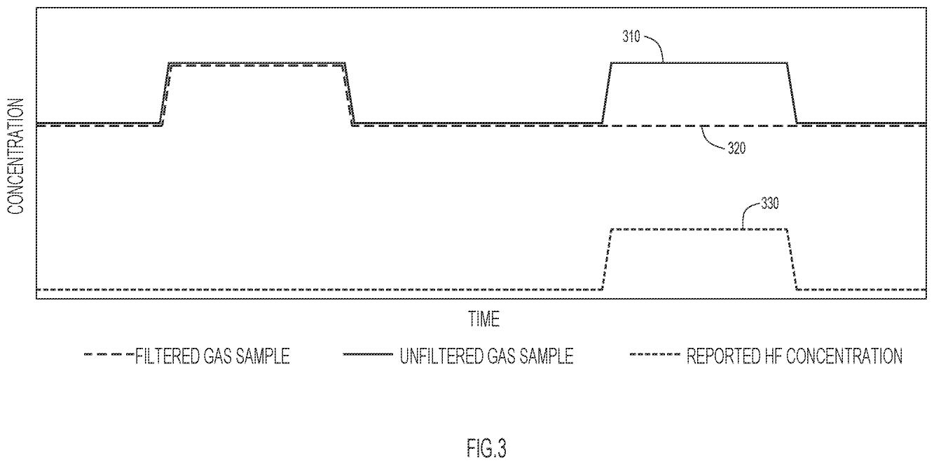

An example method of operation of the system 100 is described with reference to FIGS. 2 and 3. Referring to FIG. 2, a gas sample is collected (at 210) from an environment (e.g., a cleaning room of a semiconductor manufacturing or fabrication facility) and utilizing the system 100 of FIG. 1. Such gas sample collection can occur continuously and at a selected flow rate. A suitable inert carrier gas (e.g., substantially pure nitrogen) can be used to transport the gas sample to the mass spectrometry (MS) detector 105 for analysis. An OH⁻ reagent ion can be generated by source 170 and provided to the reaction chamber of the detector 105 to facilitate reaction with one or more compounds in the sample gas and determine concentration of analyte species containing fluorine. However, it is noted that any other one or more suitable reagent ions can also be utilized to obtain analyte fluorine species for analysis according to the methods described herein and utilizing standard or conventional SIFT-MS techniques. The unfiltered gas sample is analyzed by the detector 105 (at 220), where signal data is collected (based upon the m/z ratios of ionized analyte species of the gas sample within the detector) and such data is analyzed to obtain an indication of total fluorine species concentration within the sample, which might be considered an apparent HF concentration in the sample. In other words, in this operation this gas sample has not been scrubbed prior to MS analysis.

At some point during operation, the gas sample is diverted through the HF scrubber/filter 120 (at 230), and the filtered gas sample is then transported to the detector 105 for reaction with OH⁻ (and/or other) reagent ion(s) and subjected to MS analysis (at 240). The data from this analysis represents the sample having been filtered with certain fluorine species (primarily HF). Comparison of the sample with total fluorine species concentration (apparent HF concentration/unfiltered gas sample) and the filtered fluorine species concentration (filtered gas sample) is then performed (at 250) to determine whether HF is present in the sample and, if so, to what extent (i.e., concentration of HF within the sampled gas).

The operational steps can all be automated and selectively controlled by the processor 160 of the detector 105. Alternatively, or in combination with automated control, an operator can also manually direct performance of such operations via instructions provided to the controller of the detector 105. In addition, it is noted that the sequential order of analyzing the gas sample with total fluorine species concentration (at 220) and with filtered F⁻ concentration (at 240) is not important. In other words, data collection and MS analysis associated with the filtered gas sample can be obtained prior to data collection and MS analysis of the unfiltered gas sample (i.e., the sequential order of such data collection and analysis can be reversed).

The comparison of the signal data to determine HF concentration is depicted in the m/z data signal plots of FIG. 3. In particular, FIG. 3 shows a comparison of data signal plot 310 and data signal plot 320, both obtained by the detector, where data signal plot 310 represents the unfiltered gas sample and data signal plot 320 represents the filtered gas sample. The data signal plot 330 represents the Filtered F⁻ concentration of the filtered gas, where the data for plot 330 is obtained from a subtraction of filtered gas data signal plot 320 from unfiltered gas data signal plot 310. To state in another manner, a determination of HF concentration in the sample can be reported by the detector based upon the following analysis of signals (S):

S_{FilteredGasSample}=S_{Inteferent}+S_{background}

S_{UnfilteredGasSample}=S_{Interferent}+S_{background}+S_{HF}

S_{Reported HF Concentration}=S_{UnfilteredGasSample}−S_{FilteredGasSample}

It has been determined that such analysis effectively removes the potential for interferents to provide an accurate determination of presence and quantification of HF in a gas sample by the MS system. Further, the methods described herein can also be used in combination with other techniques, such as subtraction of a signal for an independently measured compound that is a known interferent in a particular sample.

The following example demonstrates the effectiveness of the selective HF scrubber (SHFS) MS system and corresponding method as described herein for accurately determining presence and concentration of HF in a particular environment.

### Example—Detection of HF from Gas Containing Nitrogen Trifluoride as an Interferent

Semiconductor fabrication utilizes a collection of processes such as deposition, removal, patterning, and modification, and such fabrication steps utilize a broad range of chemicals. To evaluate the performance of the selective HF scrubber (SHFS) system 100 and corresponding methods as described herein, a fab sample matrix was prepared that closely resembles an actual semiconductor fabrication environment.

Three commonly found HF-interfering fab airborne molecular contaminants (AMC) are octafluorocyclobutane (OFB) (used as an etchant and deposition gas), nitrogen trifluoride (NF₃) (used to periodically clean reaction chambers), and perfluorotributylamine (PFTBA) (used as a refrigerant).

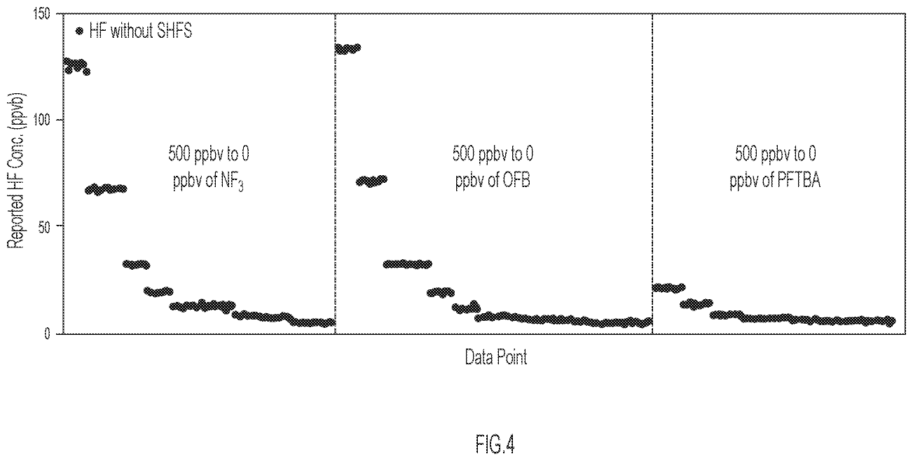

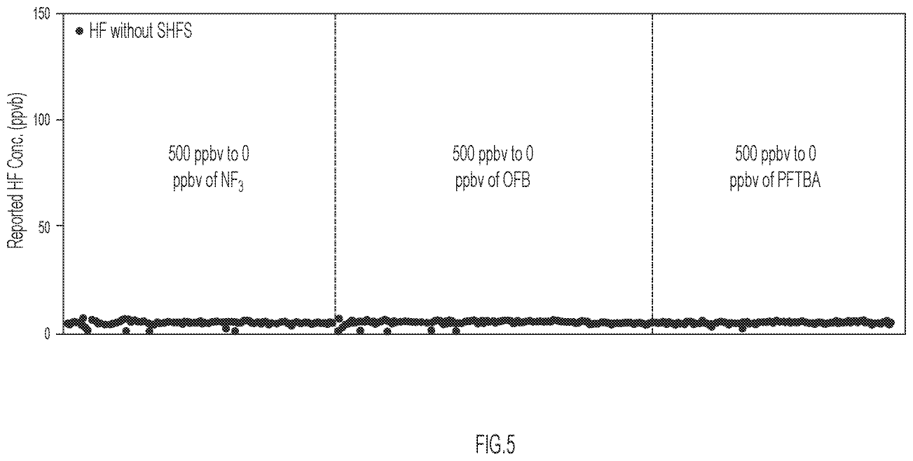

A range of process leaks were mimicked by delivering known concentrations (500 ppbv, 250 ppbv, 100 ppbv, 50 ppbv, 25 ppbv, 10 ppbv and 5 ppbv) of these three interferent gases with a constant 5 ppbv of HF. Reported concentrations of HF in the presence of these interferent compounds by system 100 without using the selective hydrogen fluoride scrubber (SHFS)/no HF scrubbing and using the SHFS/with HF scrubbing analysis are plotted in FIGS. 4 and 5, respectively. Without using the SHFS process (FIG. 4 data), it is clear that the instrument response is significantly affected by the presence of interferences. The rate of false positives is large as the sample contains high loads of interferent gases. However, when the SHFS process is used (FIG. 5 data) according to methods as described herein (and depicted in the flowchart of FIG. 2), there is a significant reduction in the rate of false positives resulting in a significantly greater accuracy in detection and quantification of HF present in the gas. In other words, the response with the SHFS (shown in FIG. 5) more accurately represents the actual HF concentration of 5 ppbv.

The following Table 3 details the results obtained for one of the interfering compounds (NF₃). It is noted that the results are similar for the other two interferents.

TABLE 3
Detailed results of the nitrogen trifluoride
(NF₃) interferent cycle with and without use of the SHFS

| | | Reported HF | Reported HF |
|---|---|---|---|
| Supplied HF | Supplied NF₃ | conc. without | conc. with |
| conc. (ppbv) | conc. (ppbv) | SHFS (ppbv) | SHFS (ppbv) |
| 5 | 5 | 4.7 | 4.4 |
| 5 | 10 | 7.7 | 4.5 |
| 5 | 25 | 12.0 | 4.6 |
| 5 | 50 | 18.4 | 4.1 |
| 5 | 100 | 31.0 | 4.7 |
| 5 | 250 | 67.2 | 4.3 |
| 5 | 500 | 126.0 | 4.0 |

Thus, the present invention facilitates accurate and reliable detection of HF as well as its quantitation in samples taken from environments in which such detection is particularly important as well as when other interfering fluorine-containing species may be present.

In an example embodiment, a mass spectrometry (MS) detection system with hydrogen fluoride (HF) specificity can comprise a MS detector to analyze a gas sample and determine a presence and/or a concentration of a plurality of chemical species within the gas sample, at least one sample flow line to facilitate flow of the gas sample from a sample source at a selected environment to an inlet of the MS detector, and a selective hydrogen fluoride scrubber coupled with the sample flow line, wherein the scrubber filters hydrogen fluoride (HF) from the gas sample prior to delivery to the inlet of the MS detector. The system can be configured to selectively permit a switch between a first flow of unfiltered gas sample to the inlet of the MS detector and a second flow of HF filtered gas sample to the inlet of the MS detector.

The scrubber can comprise a mixture of silica fibers having dimensions of no greater than 4 cm in length and transverse cross sections ranging from 15 micrometers to 25 micrometers.

The system can further comprise a diverter to selectively divert the gas sample in the sample flow line from the sample source between the first flow that bypasses the scrubber and the second flow that directs the gas sample through the scrubber.

In addition, the system can further comprise a controller that selectively controls flow of the gas sample from the sample source to the MS detector so as to facilitate delivery and analysis of the gas sample to the MS inlet in an unfiltered state via the first flow and delivery and analysis of the gas sample to the MS inlet in a HF filtered state via the second flow. The controller can further facilitate a determination of HF presence and/or HF concentration within the gas sample based upon a comparison of MS analysis of the sample gas in the unfiltered state and MS analysis of the sample gas in the HF filtered state.

The system can further comprise a reagent ion source that provides one or more reagent ions within the MS detector for exposure and reaction with one or more chemical compounds within the gas sample prior to determination of presence and/or concentration of chemical species within the MS detector. The reagent ion source can be operable to provide OH⁻ ions within the MS detector for exposure and reaction with HF present within the gas sample. In addition, the MS detector can be operable to perform Selected Ion Flow Tube-Mass Spectrometry (SIFT-MS) by exposure and reaction of a plurality of reagent ions with one or more chemical compounds within the gas sample.

In other example embodiments, a semiconductor fabrication facility can comprise a processing room that facilitates fabrication of a semiconductor component and includes the presence of hydrogen fluoride (HF), and the system as previously described herein, where the sample flow line communicates with and receives the gas sample from the processing room.

In further example embodiments, a mass spectrometry (MS) detection system with hydrogen fluoride (HF) specificity can comprise a MS detector to analyze a gas sample and determine a presence and/or a concentration of chemical species within the gas sample, a sample flow line that provides the gas sample from a selected environment to the MS detector, the sample flow line including a diverter to selectively divert the gas sample between a first sample line and a second sample line, where each of the first and second sample lines provide the gas sample to a MS inlet of the MS detector, a selective hydrogen fluoride scrubber (SHFS) provided in the second sample line, where the SHFS filters hydrogen fluoride (HF) from the gas sample prior to delivery to the inlet of the MS detector, and a controller that selectively controls the MS detector so as to facilitate delivery and analysis of the gas sample to the MS inlet in an unfiltered state via the first sample line and delivery and analysis of the gas sample to the MS inlet in a HF filtered state via the second sample line, where the controller further facilitates a determination of HF presence and/or HF concentration within the gas sample based upon a comparison of MS analysis of the sample gas in the unfiltered state and MS analysis of the sample gas in the HF filtered state.

In other example embodiments, a method for detection of hydrogen fluoride (HF) within a gas sample can comprise performing mass spectrometry (MS) analysis via a MS detector on a gas sample to obtain an unfiltered set of MS signal data, removing hydrogen fluoride (HF) from the gas sample by filtration to form a HF filtered gas sample, performing mass spectrometry (MS) analysis on the HF filtered gas sample to obtain a filtered set of MS signal data, comparing the filtered set of MS signal data with the unfiltered set of MS signal data, and determining a presence and/or a concentration of HF within the gas sample based upon the comparison.

The method can further comprise, prior to performing mass spectrometry (MS) analysis on the HF filtered gas sample, exposing and reacting one or more reagent ions with one or more chemical compounds within the gas sample to form one or more ionization products. In addition, the method can comprise generating the one or more reagent ions from a gas supply comprising oxygen, water and nitrogen. The one or more reagent ions can comprise OH⁻ ions that react with HF within the gas sample.

In the method, the MS detector can comprise a processor, and the comparing the filtered set of MS signal data with the unfiltered set of MS signal data and determining the presence and/or concentration of HF within the gas sample based upon the comparison can be performed by the processor. The method can further comprise selectively diverting, via the processor, the gas sample prior to MS analysis between a first flow line and a second flow line, where the second flow line directs the gas sample through a selective hydrogen fluoride scrubber (SHFS) prior to being directed to an inlet of the MS detector, the SHFS removes hydrogen fluoride (HF) from the gas sample by filtration, and the first flow line directs the gas sample to the inlet of the MS detector while bypassing the SHFS.

The SHFS can comprise a mixture of silica fibers having dimensions of no greater than 4 cm in length and transverse cross sections ranging from 15 micrometers to 25 micrometers.

The method can further comprise receiving the gas sample from a processing room that facilitates fabrication of a semiconductor component.

While the invention has been described in detail and with reference to specific embodiments thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

What is claimed:

1. A mass spectrometry (MS) detection system with hydrogen fluoride (HF) specificity, the system comprising:
a MS detector to analyze a gas sample and determine a presence and/or a concentration of a plurality of chemical species within the gas sample;
at least one sample flow line to facilitate flow of the gas sample from a sample source at a selected environment to an inlet of the MS detector; and
a selective hydrogen fluoride scrubber coupled with the sample flow line, wherein the scrubber filters hydrogen fluoride (HF) from the gas sample prior to delivery to the inlet of the MS detector;
wherein the system is configured to selectively permit a switch between a first flow of unfiltered gas sample to the inlet of the MS detector and a second flow of HF filtered gas sample to the inlet of the MS detector.

2. The system of claim 1, wherein the scrubber comprises a mixture of silica fibers having dimensions of no greater than 4 cm in length and transverse cross sections ranging from 15 micrometers to 25 micrometers.

3. The system of claim 1, further comprising:
a diverter to selectively divert the gas sample in the sample flow line from the sample source between the first flow that bypasses the scrubber and the second flow that directs the gas sample through the scrubber.

4. The system of claim 3, further comprising:
a controller that selectively controls flow of the gas sample from the sample source to the MS detector so as to facilitate delivery and analysis of the gas sample to the MS inlet in an unfiltered state via the first flow and delivery and analysis of the gas sample to the MS inlet in a HF filtered state via the second flow.

5. The system of claim 4, wherein the controller further facilitates a determination of HF presence and/or HF concentration within the gas sample based upon a comparison of MS analysis of the sample gas in the unfiltered state and MS analysis of the sample gas in the HF filtered state.

6. The system of claim 1, further comprising a reagent ion source that provides one or more reagent ions within the MS detector for exposure and reaction with one or more chemical compounds within the gas sample prior to determination of presence and/or concentration of chemical species within the MS detector.

7. The system of claim 6, wherein the reagent ion source is operable to provide OH-ions within the MS detector for exposure and reaction with HF present within the gas sample.

8. The system of claim 6, wherein the MS detector is operable to perform Selected Ion Flow Tube-Mass Spectrometry (SIFT-MS) by exposure and reaction of a plurality of reagent ions with one or more chemical compounds within the gas sample.

9. A semiconductor fabrication facility comprising:
a processing room that facilitates fabrication of a semiconductor component and includes the presence of hydrogen fluoride (HF); and
the system of claim 1, wherein the sample flow line communicates with and receives the gas sample from the processing room.

10. A mass spectrometry (MS) detection system with hydrogen fluoride (HF) specificity, the system comprising:
a MS detector to analyze a gas sample and determine a presence and/or a concentration of chemical species within the gas sample;
a sample flow line that provides the gas sample from a selected environment to the MS detector, the sample flow line including a diverter to selectively divert the gas sample between a first sample line and a second sample line, wherein each of the first and second sample lines provide the gas sample to a MS inlet of the MS detector;
a selective hydrogen fluoride scrubber (SHFS) provided in the second sample line, wherein the SHFS filters hydrogen fluoride (HF) from the gas sample prior to delivery to the inlet of the MS detector; and
a controller that selectively controls the MS detector so as to facilitate delivery and analysis of the gas sample to the MS inlet in an unfiltered state via the first sample line and delivery and analysis of the gas sample to the MS inlet in a HF filtered state via the second sample line, wherein the controller further facilitates a determination of HF presence and/or HF concentration within the gas sample based upon a comparison of MS analysis of the sample gas in the unfiltered state and MS analysis of the sample gas in the HF filtered state.

11. A method for detection of hydrogen fluoride (HF) within a gas sample comprising:
performing mass spectrometry (MS) analysis via a MS detector on a gas sample to obtain an unfiltered set of MS signal data;
removing hydrogen fluoride (HF) from the gas sample by filtration to form a HF filtered gas sample;
performing mass spectrometry (MS) analysis on the HF filtered gas sample to obtain a filtered set of MS signal data;
comparing the filtered set of MS signal data with the unfiltered set of MS signal data; and
determining a presence and/or a concentration of HF within the gas sample based upon the comparison.

12. The method of claim 11, further comprising:
prior to performing mass spectrometry (MS) analysis on the HF filtered gas sample, exposing and reacting one or more reagent ions with one or more chemical compounds within the gas sample to form one or more ionization products.

13. The method of claim 12, further comprising:
generating the one or more reagent ions from a gas supply comprising oxygen, water and nitrogen.

14. The method of claim 12, wherein the one or more reagent ions comprises OH-ions that react with HF within the gas sample.

15. The method of claim 11, wherein the MS detector comprises a processor, and the comparing the filtered set of MS signal data with the unfiltered set of MS signal data and determining the presence and/or concentration of HF within the gas sample based upon the comparison is performed by the processor.

16. The method of claim 15, further comprising:
selectively diverting, via the processor, the gas sample prior to MS analysis between a first flow line and a second flow line, wherein the second flow line directs the gas sample through a selective hydrogen fluoride scrubber (SHFS) prior to being directed to an inlet of the MS detector, the SHFS removes hydrogen fluoride (HF) from the gas sample by filtration, and the first flow line directs the gas sample to the inlet of the MS detector while bypassing the SHFS.

17. The method of claim 16, wherein the SHFS comprises a mixture of silica fibers having dimensions of no greater than 4 cm in length and transverse cross sections ranging from 15 micrometers to 25 micrometers.

18. The method of claim 11, further comprising:
receiving the gas sample from a processing room that facilitates fabrication of a semiconductor component.
